(19) United States
(12) Patent Application Publication
(10) Pub. No.: US 2025/0353007 A1
(43) Pub. Date: Nov. 20, 2025

(54) HIGH-SPEED IMAGING FOR MICROFLUIDIC DEVICE ANALYSIS

(71) Applicant: SAUDI ARABIAN OIL COMPANY, Dhahran (SA)

(72) Inventors: Abdulrahman ALJEDAANI, Dhahran (SA); Abdulaziz ALREFAEI, Dhahran (SA); Ahmed A. ALZAHRANI, Dhahran (SA)

(73) Assignee: SAUDI ARABIAN OIL COMPANY, Dhahran (SA)

(21) Appl. No.: 18/663,451

(22) Filed: May 14, 2024

(51) Int. Cl. B01L 3/00; G06T 7/20

(52) U.S. Cl. CPC B01L 3/502746; G06T 7/20; B01L 2200/0647; B01L 2300/0874; B01L 2300/1838; G06T 2207/30181

(57) ABSTRACT

A system for simulating flow within carbonate rock under reservoir conditions includes a substantially two-dimensional microfluidics device defining a flowpath therethrough, the substantially two-dimensional microfluidics device including a first wall comprising thin slices of carbonate rock, an opposing wall comprising transparent glass, a plurality of surrounding walls, and a throat defined within the flowpath to simulate flow through a porous structure. The system further includes a high-speed camera at or near the opposing wall and aimed at the first wall and the throat of the substantially two-dimensional microfluidics device, the high-speed camera operable to capture images and/or videos of fluid flow through the substantially two-dimensional microfluidics device.

### FIELD OF THE DISCLOSURE

The present disclosure relates generally to experimentally simulating fluid interaction, and, more particularly, to systems and methods for microfluidic device analysis of simulated reservoir conditions.

### BACKGROUND OF THE DISCLOSURE

Hydrocarbon reservoirs commonly include high pressures and temperatures which can affect the permeability and porosity of surrounding rock formations. The dynamic nature of hydrocarbon reservoirs and surrounding carbonate rock can lead to a variety of solid-liquid and liquid-liquid interactions therein. Further complicating the dynamics within hydrocarbon reservoirs, the wettability of carbonate rocks can alter flow characteristics and geological parameters based upon these solid-liquid interactions. Accordingly, to understand the finer details of fluid interactions in a hydrocarbon reservoir, techniques for simulation of reservoir conditions are desired.

Microfluidics devices have been developed to simulate micron-scale pore throats within carbonate rocks and to visualize the dynamic flow patterns therethrough. Microfluidics devices can utilize these small-scale experimental simulations to glean insights into the fluid dynamics of hydrocarbon reservoirs on a macro-scale. However, due to the combination of fluid interactions and the variation of geological parameters with wettability, quantitative measurement and analysis of microfluidics devices can be challenging. Further, for an experimental set-up to mimic reservoir conditions, velocities may be required within the microfluidics devices that are difficult to accurately capture. While these velocities can be low enough to form laminar flows or viscous-dominated Stokes flows, the micron-scale sizing of microfluidics can present a unique challenge for data collection even at relatively low velocities.

### SUMMARY OF THE DISCLOSURE

Various details of the present disclosure are hereinafter summarized to provide a basic understanding. This summary is not an exhaustive overview of the disclosure and is neither intended to identify certain elements of the disclosure, nor to delineate the scope thereof. Rather, the primary purpose of this summary is to present some concepts of the disclosure in a simplified form prior to the more detailed description that is presented hereinafter.

According to an embodiment consistent with the present disclosure, a system for simulating flow within carbonate rock under reservoir conditions includes a substantially two-dimensional microfluidics device defining a flowpath therethrough, the substantially two-dimensional microfluidics device including a first wall comprising thin slices of carbonate rock, an opposing wall comprising transparent glass, a plurality of surrounding walls, and a throat defined within the flowpath to simulate flow through a porous structure. The system further includes a high-speed camera at or near the opposing wall and aimed at the first wall and the throat of the substantially two-dimensional microfluidics device, the high-speed camera operable to capture images and/or videos of fluid flow through the substantially two-dimensional microfluidics device.

In another embodiment, a system for simulating flow out of carbonate rock under reservoir conditions includes a three-dimensional microfluidics device defining a flowpath therethrough, the three-dimensional microfluidics device including a plurality of walls including the flowpath, a packed bed of spheres within the plurality of walls and including a plurality of spheres of a carbonate rock composition, an interim fluid conduit in fluid communication with an outlet of the flowpath, and a secondary flow channel formed of transparent glass plates and in fluid communication with the interim fluid conduit to receive flow from the flowpath. The system further includes a high-speed camera at or near the secondary flow channel and aimed therethrough, the high-speed camera operable to capture images and/or videos of fluid flow out of the flowpath and packed bed and through the secondary flow channel.

In a further embodiment, a computer-implemented method for observing flow through a microfluidics device in reservoir conditions includes heating a flow environment within the microfluidics device to simulate a reservoir temperature within a flow environment of the microfluidics device, pumping a working fluid at a specified pressure to simulate a reservoir pressure within the flow environment, initiating, via a high-speed camera aimed at or near the flow environment, imaging of a flow through and/or out of the flow environment, and recording, via a computer-readable storage medium, images and/or videos obtained via the high-speed camera for analysis, wherein at least one wall of the microfluidics device includes a superhydrophobic coating applied thereon.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

FIG. 1 is an example schematic view of a substantially two-dimensional microfluidics device with a transparent design for simulating reservoir conditions.

FIG. 2 is an example schematic view of a microfluidics system for high-speed capture of flow within the substantially two-dimensional microfluidics device, according to at least one embodiment of the present disclosure.

FIG. 3 is an example a three-dimensional microfluidics device with a packed bed of carbonate rock for simulating reservoir conditions.

FIG. 4 is an example schematic view of a microfluidics system for high-speed capture of flow out of the three-dimensional microfluidics device, according to at least one embodiment of the present disclosure.

FIG. 5 is an example of a method for simulating and capturing flow under reservoir conditions in a substantially two-dimensional microfluidics device.

FIG. 6 is an example of a method for simulating and capturing flow out of a three-dimensional microfluidics device in reservoir conditions.

FIG. 7 is a block diagram of a computer system that may be used to implement one or more of the systems or methods described herein in accordance with certain embodiments.

### DETAILED DESCRIPTION

Embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the claimed subject matter. However, it will be apparent to one of ordinary skill in the art that the embodiments disclosed herein may be practiced without these specific details. In other instances, well-known features have not been described in detail to avoid unnecessarily complicating the description. Additionally, it will be apparent to one of ordinary skill in the art that the scale of the elements presented in the accompanying Figures may vary without departing from the scope of the present disclosure.

Embodiments in accordance with the present disclosure generally relate to experimentally simulating fluid interaction, and, more particularly, to systems and methods for microfluidic analysis of simulated reservoir conditions. Embodiments herein can include both substantially two-dimensional and three-dimensional microfluidics devices, systems to facilitate operation thereof, and methods of operation. The microfluidics devices can be coupled with high-speed cameras to capture images and/or videos of the fluid flow through or out of the devices. The high-speed cameras can capture images at frame rates up to about 1,000,000 frames per second to visualize and analyze fluid-solid and fluid-fluid interactions of specific particles or parcels as they travel through transparent components of the devices. The microfluidic devices can include carbonate rock structures therein, such as a rear wall of the substantially two-dimensional device formed of thin slices of carbonate rock. The three-dimensional microfluidics device can similarly include a matrix, or packed bed, of carbonate rock spheres sintered or compacted to obstruct a flowpath and simulate a hydrocarbon reservoir rock formation.

The embodiments disclosed herein can enable the rapid capture of flow images within and out of microfluidics devices to analyze the fluid-fluid and fluid-solid interactions occurring within a hydrocarbon reservoir. Components can be provided which enable tuning and control of both temperature and pressure within the flow to mimic the conditions of a hydrocarbon reservoir. In some embodiments, transparent glass plates can be used in construction of the devices and flowpaths, and these transparent glass plates can include a superhydrophobic coating applied thereon. As such, only the fluid-fluid interactions, and fluid-solid interactions with the carbonate rock, can be observed and studied. Varying light sources can be provided to enable imaging through visible light and infrared light depending on the composition of the working fluid. Supplemental particles can be additionally provided to the flow to enable studying of changes to geophysical properties and wetting behaviors of the carbonate rock used in the devices. The devices, systems, and methods disclosed herein can mimic the conditions existing within a hydrocarbon reservoir in both substantially 2D and 3D experimental setups, and can enable high-speed imaging to analyze the microscopic and rapid interactions occurring within.

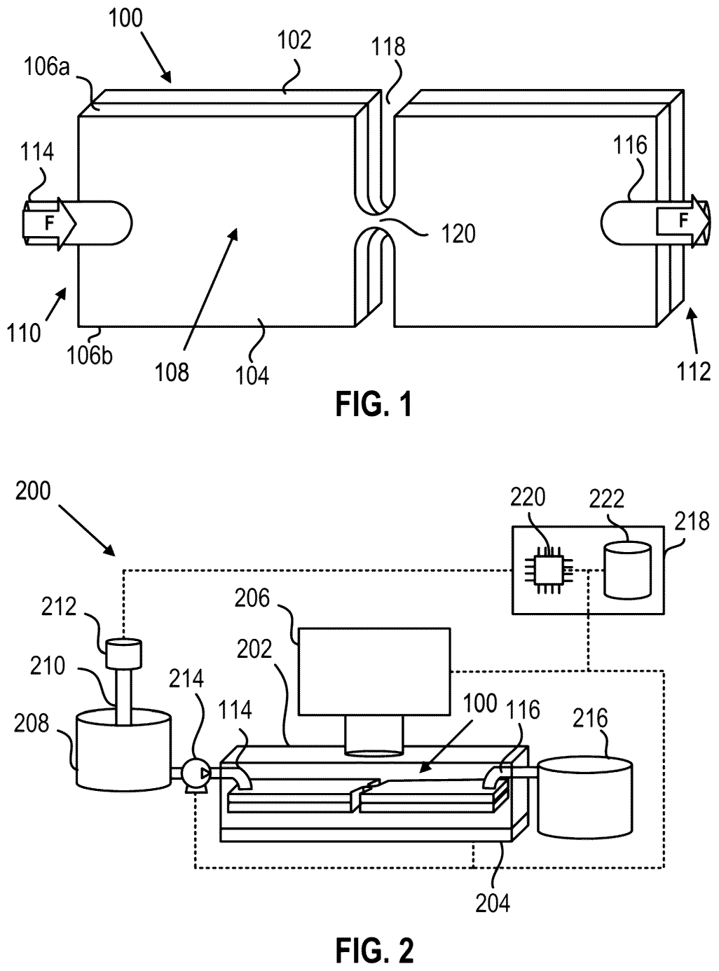

FIG. 1 is an example schematic view of a substantially two-dimensional microfluidics device 100 with a transparent design for simulating reservoir conditions. The substantially two-dimensional microfluidics device 100 (hereinafter, “the 2D device 100”) can include a rear wall 102 formed of slices of carbonate rock. The 2D device 100 can be considered “substantially two-dimensional”, such that the thickness of the 2D device 100 and any flowpaths therein are negligible when compared to the width and length of the 2D device 100. While the 2D device 100 can include a three-dimensional flowpath therein, the “substantially two-dimensional” nature of the 2D device 100 enables study of two-dimensional fluid dynamics without accounting for a third flow direction during analysis. The carbonate rock of the rear wall 102 can range in thickness from about 100 mm to about 500 mm in thickness, which can affect the wettability and parameters of the carbonate rock. In some embodiments, the carbonate rock of the rear wall 102 can be formed of limestone (CaCO₃) or dolomite (CaMg(CO₃)₂). In these embodiments, the carbonate rock of the rear wall 102 can be interchangeable within the 2D device 100, such that a variety of permeability and porosity values can be simulated. The 2D device 100 can further include a front wall 104 formed of a transparent glass plate, through which fluid interactions with the carbonate rock of the rear wall 102 can be observed. Similarly, the 2D device 100 can include a top wall 106a and a bottom wall 106b formed of a transparent glass plate.

The 2D device 100 can define a thin, substantially two-dimensional flowpath 108 between the rear wall 102, front wall 104, and top and bottom wall 106a-b. The substantially two-dimensional flowpath 108 may be formed in a micron-scale thickness, such that depth-related flow characteristics are effectively nullified, as described above. The substantially two-dimensional flowpath 108 can flow from a first end 110 to a second end 112 of the 2D device 100, such that a flow direction is established for visualization and data collection. The substantially two-dimensional flowpath 108 can receive a fluid “F” from a fluid inlet 114 defined at or near the first end 110 of the 2D device 100. The fluid “F” can flow across the 2D device 100, and can be received in a fluid outlet 116 defined at or near the second end 112. In some embodiments, the fluid “F” can be a water-in-oil emulsion to mimic fluid conditions within a hydrocarbon reservoir. In these embodiments, the flow of the fluid “F” from the fluid inlet 114 to the fluid outlet 116 across the substantially two-dimensional flowpath 108 can enable visualization and analysis of hydrocarbon fluid interactions with the carbonate rock of the rear wall 102, as well as the pinning of contact lines inside the multiphase flow.

To aid in the visualization and analysis of hydrocarbon fluid interactions in a reservoir, one or more insets 118 can be defined within the 2D device 100. The insets 118 can vary in shape and size, such that the 2D device 100 may be manufactured to include a desired throat 120 within the substantially two-dimensional flowpath 108. The throat 120 can represent flow within and between porous structures in a hydrocarbon reservoir, such as the voids defined between carbonate rock particles. Accordingly, a variety of insets 118 can be utilized in representing different thicknesses and shapes of the throat 120 to generate data for any fluid restriction or obstruction, and can further introduce a shearing flow within the substantially two-dimensional flowpath 108.

In some embodiments, each of the front wall 104 and the top and bottom walls 106a-b can be made superhydrophobic during manufacturing. The application of a superhydrophobic coating to the glass of the front wall 104 and the top and bottom walls 106a-b may limit any interaction between the fluid “F” and the structure of the 2D device 100 excepting the carbonate rock of the rear wall 102. In further embodiments, a chemical coating can be applied to the glass of the front wall 104 and the top and bottom walls 106a-b to mimic chemical interactions with surfaces of a reservoir. Accordingly, any analysis and visualization of flow in the 2D device 100 can be limited to fluid-solid interactions with the carbonate rock of the rear wall 102, the fluid-fluid interaction within the water-in-oil emulsion, and shearing interaction with the throat 120.

FIG. 2 is an example schematic view of a microfluidics system 200 for high-speed capture of flow within the substantially two-dimensional microfluidics device 100, according to at least one embodiment of the present disclosure. In some embodiments, the microfluidics system 200 (hereinafter, “the system 200”) can include the 2D device 100 within a simulation chamber 202. The simulation chamber 202 can provide an enclosed area for controlling an ambient temperature of the 2D device 100 during operation, as well as a pressurized environment to mimic reservoir conditions. The simulation chamber 202 can be formed of a transparent material, such as a glass or an acrylic, such that imaging may be performed therethrough. In these embodiments, a heater 204 can be mounted on a side of the simulation chamber 202 to provide an influx of heat and maintain a desired temperature therein. In further embodiments, however, the simulation chamber 202 may be omitted, and the heater 204 may be mated directly to the 2D device 100.

The system 200 can further include a high-speed camera 206 at or near a surface of the simulation chamber 202 or the 2D device 100. The high-speed camera 206 can capture images and videos of the flow through the 2D device 100 at rates ranging from about 25,000 frames per second to about 1,000,000 frames per second, depending on resolution size of the images. The high-speed camera 206 may be focused on the throat 120 of FIG. 1 to specifically capture flow patterns through the restriction formed in the 2D device 100, while further capturing images of the fluid-solid interaction with the carbonate rock of the rear wall 102 of FIG. 1. The transparent nature of the 2D device 100 and the simulation chamber 202 can enable imaging therethrough via the high-speed camera 206. In a laminar flow with a velocity of approximately one meter per second, a fluid particle can travel one micron in one micro-second. Accordingly, the use of the high-speed camera 206 can enable accurate visualization of fluid-solid interactions that is not possible with traditional imaging techniques.

Within the system 200, the fluid inlet 114 of the 2D device 100 can be in fluid communication with a source tank 208. The source tank 208 can provide the fluid “F” to the system 200 for visualization of the flow through the 2D device 100. As discussed above, the fluid “F” stored in the source tank 208 can include a water-in-oil emulsion to mimic fluid conditions within a hydrocarbon reservoir. However, to accurately simulate reservoir conditions, a plurality of additional particles can be supplemented into the fluid “F” to represent further rock and mineral components present within a hydrocarbon reservoir. As such, in some embodiments, the source tank 208 can be in further communication with a solids conduit 210. The solids conduit 210 can be in communication with a particle emulsion tank 212, which may selectively output one or more particle types into the particle conduit 210 and the source tank 208. The particle emulsion tank 212 can include supplemental particles including, but not limited to, calcite, quartz, anhydrate, and any combination thereof. The dissolution and interaction of these supplemental particles against the carbonate rock of the rear wall 102 of FIG. 1 can affect wettability and other geological parameters thereof.

The system 200 can further include at least one pump 214 interposing the fluid inlet 114 and the source tank 208. The pump 214 can provide the fluid “F” and any supplemental particles to the 2D device 100 at a desired pressure or flowrate to further mimic reservoir conditions. The pump 214 can accordingly maintain the velocity of the fluid “F” through the throat 120 of FIG. 1, and can be tuned as needed to produce various flow scenarios. As the fluid “F” flows throughout the 2D device and into the fluid outlet 116, the fluid “F” may be disposed of in a waste tank 216 in fluid communication with the fluid outlet 116. The waste tank 216 can receive the fluid “F” and any supplemental particles for eventual disposal or recycling. In some embodiments, the waste tank 216 can be in fluid communication with the source tank 208 and recycling equipment (not shown) to replenish the fluid “F” to the source tank 208 and the supplemental particles to the particle emulsion tank 212.

The flow of the fluid “F” through the 2D device 100, and the conditions affecting the carbonate rock of the rear wall 102 of FIG. 1 can be directly affected by the temperature and pressure of the fluid “F” and environment of the 2D device 100. Accordingly, the heater 204 and the pump 214 can be operably coupled to a computing device 218 for controlling and monitoring the system 200. The computing device 218 can include a processor 220 and a computer-readable storage medium 222, and can be in physical, wired communication with the rest of the system 200. The computing device 218 can include any computing device, for example, a desktop computer, a server, a controller, a blade, a mobile phone, a tablet, a laptop, a personal digital assistant (PDA), or other types of portable (or stationary) devices. By way of example, the computer-readable storage medium 222 can be implemented, for example, as a non-transitory computer storage medium, such as volatile memory (e.g., random access memory), non-volatile memory (e.g., a hard disk drive, a solid-state drive, a flash memory, or the like), or a combination thereof. The processor 220 can be implemented, for example, as one or more processor cores. The computer-readable storage medium 222 can store machine-readable instructions for control and monitoring of the system 200 that can be retrieved and executed by the processor 220.

Each of the processor 220 and the computer-readable storage medium 222 can be implemented on a similar or a different computing platform. The computing platform could be implemented in a computing cloud and thus on a cloud computing architecture. In such a situation, features of the computing platform could be representative of a single instance of hardware or multiple instances of hardware executing across the multiple of instances (e.g., distributed) of hardware (e.g., computers, routers, memory, processors, or a combination thereof). Alternatively, the computing platform could be implemented on a single dedicated server or workstation. In further embodiments, the computing device 218 can be in wireless communication with the rest of the system 200, and can be locally stored, or accessed as a cloud device over the internet.

The computing device 218 can be in further communication with the high-speed camera 206, such that the images or videos can be provided to the computing device 218 in real-time. Through analysis of one or more of the images or videos during operation, the computing device 218 can adjust the heater 204 and the pump 214 to achieve flow conditions mimicking reservoir conditions. Further, the computer-readable storage medium 222 can store the images and videos from the high-speed camera 206 for further analysis and use following the experimental runs of the system 200. In some embodiments, the computing device 218 can be in further communication with the particle emulsion tank 212, or a flow control component/valve thereof. In these embodiments, the computing device 218 can modify a flow of the supplemental particles into the source tank 208 as desired to test for changes in wettability and fluid interactions.

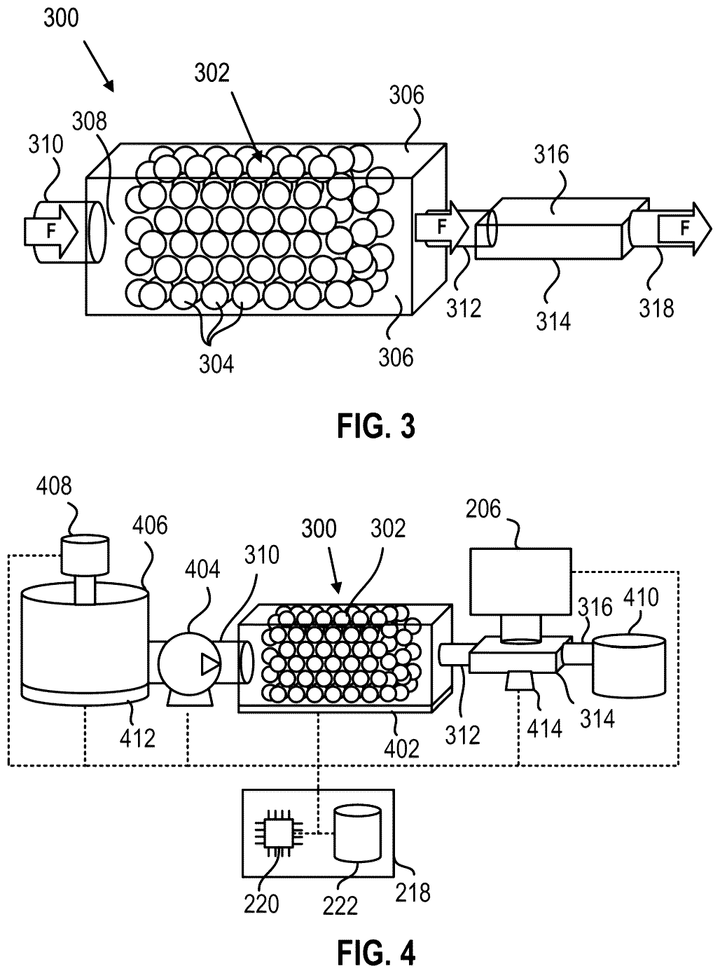

FIG. 3 is an example a three-dimensional microfluidics device 300 with a packed bed, or matrix, 302 of carbonate rock for simulating reservoir conditions. The packed bed 302 can include a plurality of sintered or compacted spheres 304 formed of calcium-carbonate or from crushed carbonate rocks and powders. The packed bed 302 and the spheres 304 comprising it can simulate conditions within a hydrocarbon reservoir. The three-dimensional microfluidics device 300 (hereinafter, “the 3D device 300) can include a plurality of glass plates 306 surrounding the packed bed 302 and defining a flowpath 308 surrounding the spheres 304. The fluid “F” can be passed through the flowpath 308 and the packed bed 302 to simulate the fluid-solid interactions that can occur in a hydrocarbon reservoir. The fluid “F” can be introduced to the flowpath 308 via a fluid inlet 310 at a rate to maintain a desired pressure within the packed bed 302.

In contrast to the 2D device 100 of FIGS. 1-2, the 3D device 300 can prevent direct imaging of the fluid interactions within the packed bed 302, as the spheres 304 can be formed of opaque rock. As such, the 3D device 300 can include an interim conduit 312 on an opposing side of the packed bed 302 from the fluid inlet 310. The interim conduit 312 can fluidly couple the flowpath 308 through the packed bed 302 with a secondary flow channel 314. The secondary flow channel 314 can include a plurality of transparent glass plates 316 to form the secondary flow channel 314 with a thickness of about 100 mm. The secondary flow channel 314 can receive the fluid “F” from the flowpath 308 following the fluid-solid interactions within the packed bed 302. The secondary flow channel 314 can be fully transparent such that imaging may be performed through the secondary flow channel 314 to assess the effects of differing flow conditions and compositions. The secondary flow channel 314 can be in further fluid communication with a fluid outlet 318 that can transport the flow out of the 3D device 300. As with the 2D device 100, the plurality of glass plates 306 of the 3D device 300, as well as of the secondary flow channel 314, can include a superhydrophobic coating thereon. The superhydrophobic coating can prevent fluid interactions with the walls of the simulated environment, such that only the fluid interactions with the packed bed 302 can be analyzed.

FIG. 4 is an example schematic view of a microfluidics system 400 for high-speed capture of flow out of the three-dimensional microfluidics device 300, according to at least one embodiment of the present disclosure. The microfluidics system 400 (hereinafter, “the system 400”) can include the high-speed camera 206 of FIG. 2, such that the same high-speed camera technology can be utilized in capturing fluid flow within the secondary flow channel 314. As discussed above, the high-speed camera 206 can capture images and videos of fluid emulsion “F” downstream of the packed bed 302 to monitor the composition thereof. While direct imaging of fluid interactions within the packed bed 302 can be omitted in the system 400, the effects of different flow conditions and fluid additives on droplet size distribution in the fluid “F” over time can be monitored in the secondary flow channel 314.

Accordingly, the system 400 can include a heater 402 mated to the 3D device 300 to tune the temperature of the environment within the packed bed 302 to match reservoir conditions. Similarly, a pump 404 can be fluidly coupled to the fluid inlet 310 to enable fine-tuning of pressures within the packed bed 302. The pump 404 can interpose a fluid line connecting the fluid inlet 310 and a fluid source 406. The fluid source 406 can provide the fluid “F” to the 3D device 300 as needed, and can maintain a mixed state within the water-in-oil emulsion. As with the system 200 of FIG. 2, the system 400 can include one or more particle emulsion tanks 408 in fluid communication with the fluid source 406. The particle emulsion tanks 408 can contain a plurality of supplemental particles to be introduced to the fluid “F” for altering wettability and geological properties of the packed bed 302. The particle emulsion tanks 408 can include one or more flow components or valves (not shown) that can control a flow of the supplemental particles into the fluid source 406. The system 400 can further include a waste tank 410 in fluid communication with the fluid outlet 318 of the 3D device 300. The waste tank 410 can receive outflow from the secondary flow channel 314, and can include storage for the fluid “F” expelled therefrom. The waste tank 410 can be utilized in storing this fluid “F” for later disposal, recycling, or analysis to determine any compositional changes observed by the high-speed camera 206.

In some embodiments, the system 400 can further include a source heater 412 mated to the fluid source 406. The source heater 412 can provide an influx of heat to maintain a desired temperature within the fluid source 406, such that reservoir conditions are maintained in the 3D device 300 and the fluid “F” in general. Further, to aid in imaging of the fluid “F” within the secondary flow channel 314, the system 400 can include a light source 414 at or near the secondary flow channel 314. The light source 414 can include both visible light sources and infrared light sources to enhance imaging within the secondary flow channel 314. In some embodiments, the light source 414 can utilize a combination of visible and infrared light to enable imaging through both water and crude oil during operations.

As in the system 200 of FIG. 2, the system 400 can include a control unit in the form of the computing device 218. The computing device 218 can include the processor 220 and the computer-readable storage medium 222, and can be in physical, wired communication with the rest of the system 400. The computing device 218 can be in direct communication with the heater 402, the pump 404, the source heater 412, the particle emulsion tank 408, the light source 414, and the high-speed camera 206 to control the flow conditions and imaging thereof. The computing device 218 can autonomously control, or can be utilized by an operator to control, operations of the system 400 and flow through the 3D device 300.

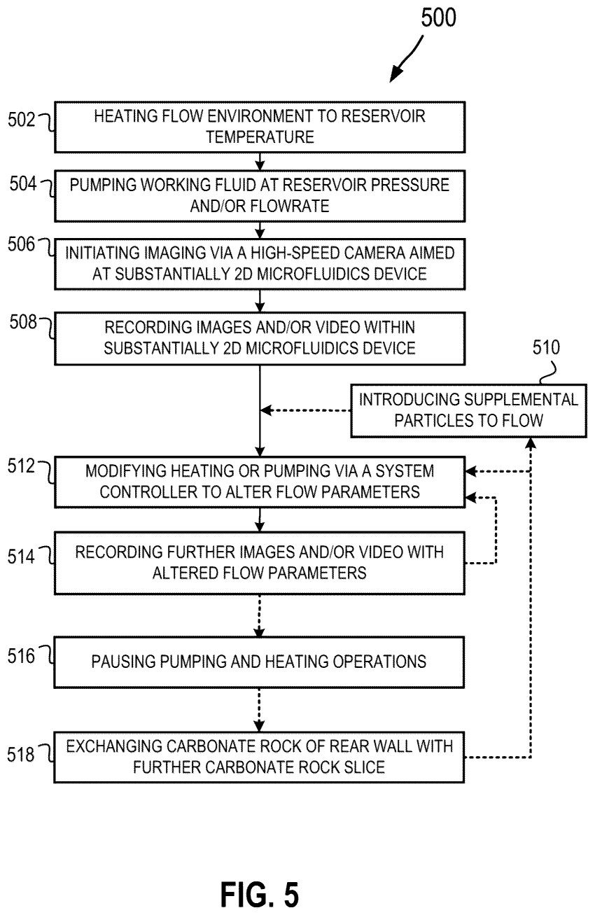

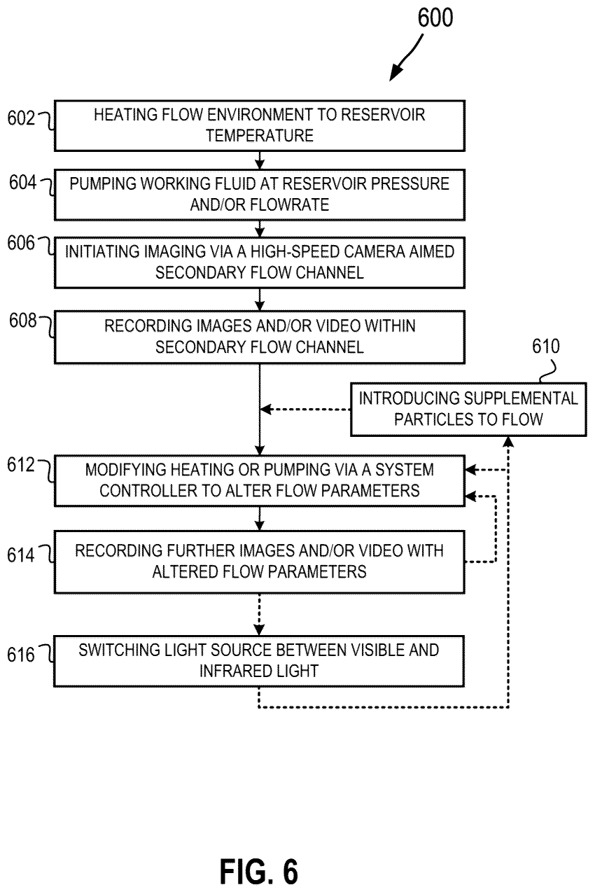

In view of the structural and functional features described above, example methods will be better appreciated with reference to FIGS. 5-6 While, for purposes of simplicity of explanation, the example methods of FIGS. 5-6 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement the methods, and conversely, some actions may be performed that are omitted from the description.

FIG. 5 is an example of a method 500 for simulating and capturing flow under reservoir conditions in a substantially two-dimensional microfluidics device. The method 500 can be implemented by the 2D device 100 and/or the system 200, as shown in FIGS. 1-2. Thus, reference can be made to the example of FIGS. 1-2 in the example of FIG. 5. The method 500 can begin at 502 with heating a flow environment for a working fluid (e.g., the fluid “F”) to a temperature simulating that of a hydrocarbon reservoir. The heating at 502 can be performed by a heater (e.g., the heater 204) to bring a flow environment to the desired temperature. The heater can be operably coupled to the flow environment, such that the heater can directly provide heat to the flow environment at 502.

The flow environment of interest can be a substantially 2D device (e.g., the substantially two-dimensional microfluidics device 100) or a simulation chamber (e.g., the simulation chamber 202) in which the 2D device can be installed. The 2D device can define a flowpath (e.g., the flowpath 108) through which the working fluid can pass. The 2D device can include a rear wall (e.g., the rear wall 102) formed of one or more thin slices of carbonate rock, while remaining walls of the 2D device can be formed of clear glass plates. In some embodiments, the clear glass plates can include a superhydrophobic coating thereon such that the working fluid is repelled from all surfaces excepting the rear wall.

The method 500 can further include pumping the working fluid at a pressure and/or flowrate at 504 to further simulate the working conditions of a hydrocarbon reservoir. The pumping at 504 can be performed via a pump (e.g., the pump 214) in fluid communication with a fluid inlet (e.g., the fluid inlet 114) of the 2D device. The pump can provide the working fluid to the 2D device at the desired pressure to simulate reservoir conditions, and can be in further fluid communication with a fluid source (e.g., the source tank 208) for supplying the working fluid. In some embodiments, the working fluid can be a water-in-oil emulsion for mimicking a working fluid in a hydrocarbon reservoir.

With flow initiated within the 2D device, the method 500 can further include initiating imaging via a high-speed camera (e.g., the high-speed camera 206) aimed at the 2D device through the clear glass plates of the flow environment. The high-speed camera can be placed directly at or near the flow environment, whether that is the simulation chamber or the 2D device itself. The high-speed camera can be initiated via a system controller (e.g., the computing device 218) which can also be utilized at 502 and 504 for controlling the heater and the pump. The method 500 can continue at 508 with recording images and/or video of the flow within the 2D microfluidics device. The recording at 508 can include recording at high frame rates, ranging from about 25,000 frames per second to about 1,000,000 frames per second, depending on resolution size of the images recorded. The high frame rate of the recording at 508 can enable tracking and analysis of fluid particles or parcels as they undergo fluid-fluid and fluid-solid interactions within the 2D device. The images and videos recorded at 508 can be stored on the system controller, such that a computer-readable storage medium (e.g., the computer-readable storage medium 222) can store the images and videos for later analysis.

In some embodiments, the method 500 can include introducing supplemental particles to the flow within the 2D device at 510. The supplemental particles can be stored in a particle emulsion tank (e.g., the particle emulsion tank 212) and can be introduced to the fluid source or to the fluid inlet of the 2D device directly. The supplemental particles can include, but are not limited to, calcite, quartz, anhydrate, and any combination thereof. The dissolution and interaction of these supplemental particles against the carbonate rock of the rear wall can affect wettability and other geological parameters thereof. Accordingly, introducing the supplemental particles at 510 can create a new flow environment and parameters, such that further analysis can be performed.

The method 500 can further include modifying the heating or pumping within the system via the system controller at 512 to alter the flow parameters. As with the introduction of supplemental particles at 510, the modifications made at 512 can create a new flow environment for further study. The modifications made at 512 can represent a new hydrocarbon reservoir, or can further represent a dynamic event therein for analysis. As such, the method 500 can include recording further images and/or video at 514 with the altered flow parameters. The further recording performed at 514 can be utilized in analysis of the new flow parameters, as well as in analyzing the direct effects of the modifications made at 512, the supplemental particles introduced at 510, and any combination thereof. The analysis of these effects can enable greater understanding of the reservoir environment and the dynamic nature of the flow structures therein.

In some embodiments, the method 500 can include pausing the heating and pumping operations at 516 to effectively stop simulation of the reservoir environment in the 2D device. The pausing at 516 can further include pausing of the imaging within the system, as modifications can be performed to the components of the system. In these embodiments, the method 500 can continue at 518 with exchanging of the carbonate rock of the rear wall with a further slice of carbonate rock. The flow studies performed within the 2D device can be used with a variety of carbonate rock samples, and during operation the slices of carbonate rock included in the rear wall can be exchanged. For example, a limestone layer of carbonate rock can be removed from the rear wall of the 2D device and replaced with a dolomite layer to simulate differing reservoir conditions and compositions. Accordingly, the method 500 can continue at 512 with any further modifications to the flow parameters before further image recording is performed at 514. The method 500 can be performed cyclically, such that changes to flow parameters, introductions of different supplemental particles, and exchanging of carbonate rock slices can be performed, and the differences in the flow patterns and interactions can be analyzed and studied.

FIG. 6 is an example of a method 600 for simulating and capturing flow out of a three-dimensional microfluidics device in reservoir conditions. The method 600 can be implemented by the 3D device 300 and/or the system 400, as shown in FIGS. 3-4. Thus, reference can be made to the example of FIGS. 3-4 in the example of FIG. 6. The method 600 can begin at 602 with heating a flow environment for a working fluid (e.g., the fluid “F”) to a temperature simulating that of a hydrocarbon reservoir. The heating at 502 can be performed by a heater (e.g., the heater 402 and/or the source heater 412) to bring the flow environment to the desired temperature. The heater can be operably coupled to the flow environment and/or to a fluid source (e.g., the fluid source 406), such that flow environment can be maintained at a desired heat at 502.

The flow environment of interest can be a 3D device (e.g., the three-dimensional microfluidics device 300). The 3D device can define a flowpath (e.g., the flowpath 308) through a packed bed (e.g., the packed bed 302) through which the working fluid can pass. The packed bed can consist of a plurality of spheres (e.g., the plurality of spheres 304) formed of a crushed, sintered, or shaped calcium-carbonate, or other carbonate rock components. The walls of the 3D device can be formed of clear glass plates, such that the packed bed can be seen through the 3D device for visual inspection. In some embodiments, the clear glass plates can include a superhydrophobic coating thereon such that the working fluid is repelled from all surfaces excepting the packed bed.

The method 500 can further include pumping the working fluid at a pressure and/or flowrate at 604 to further simulate the working conditions of a hydrocarbon reservoir. The pumping at 604 can be performed via a pump (e.g., the pump 404) in fluid communication with a fluid inlet (e.g., the fluid inlet 310) of the 3D device. The pump can provide the working fluid to the 3D device at the desired pressure to simulate reservoir conditions, and can be in further fluid communication with the fluid source for supplying the working fluid. In some embodiments, the working fluid can be a water-in-oil emulsion for mimicking a working fluid in a hydrocarbon reservoir.

With flow initiated within the 3D device, the method 500 can further include initiating imaging via a high-speed camera (e.g., the high-speed camera 206) aimed at a secondary flow channel (e.g., the secondary flow channel 314) of the 3D device. The high-speed camera can capture fluid composition and flow information through the clear glass plates of the secondary flow channel. As with the clear glass plates discussed above, the clear glass plates of the secondary flow channel can similarly include a superhydrophobic coating thereon. The high-speed camera can be placed directly at or near the secondary flow channel, such that flow out of the packed bed can be captured. The high-speed camera can be initiated via a system controller (e.g., the computing device 218) which can also be utilized at 602 and 604 for controlling the heater and the pump. The method 600 can continue at 608 with recording images and/or video of the flow out of the 3D microfluidics device and into the secondary flow channel

In some embodiments, the method 600 can include introducing supplemental particles to the flow within the 3D device at 610. The supplemental particles can be stored in a particle emulsion tank (e.g., the particle emulsion tank 408) and can be introduced to the fluid source or to the fluid inlet of the 3D device directly. The supplemental particles can include, but are not limited to, calcite, quartz, anhydrate, and any combination thereof. The dissolution and interaction of these supplemental particles against the carbonate rock of the rear wall can affect wettability and other geological parameters thereof. Accordingly, introducing the supplemental particles at 610 can create a new flow environment and parameters, such that further analysis can be performed. The composition of the resulting outflow can be captured in the secondary flow channel to further determine absorption and obstruction of the supplemental particles within the packed bed during flow.

The method 600 can further include modifying the heating or pumping within the system via the system controller at 612 to alter the flow parameters. The modifications made at 612 can represent a new hydrocarbon reservoir, or can further represent a dynamic event therein for analysis. As such, the method 600 can include recording further images and/or video at 614 with the altered flow parameters. The further recording performed at 614 can be utilized in analysis of the new flow parameters, as well as in analyzing the direct effects of the modifications made at 612, the supplemental particles introduced at 610, and any combination thereof. The analysis of these effects can enable greater understanding of the reservoir environment and the dynamic nature of the flow structures therein.

In some embodiments, the method 600 can include altering or switching a light source (e.g., the light source 414) between visible and infrared light at 616. The light source can be mounted at or near the secondary flow channel to provide lighting through the working fluid “F” during imaging. Visible light provided by the light source can be utilized in imaging of, and through, water in the secondary flow channel. In contrast, the infrared light provided by the light source can be utilized in imaging of, and through, crude oil in the secondary flow channel. Accordingly, the modification of the lighting at 616 can alter the analysis performed within the secondary flow channel and the imaging of interest to be performed. The method 600 can be performed cyclically, such that changes to flow parameters, introductions of supplemental particles, and variations to the wavelengths of provided light, can be performed, and the differences in the flow patterns and interactions can be analyzed and studied.

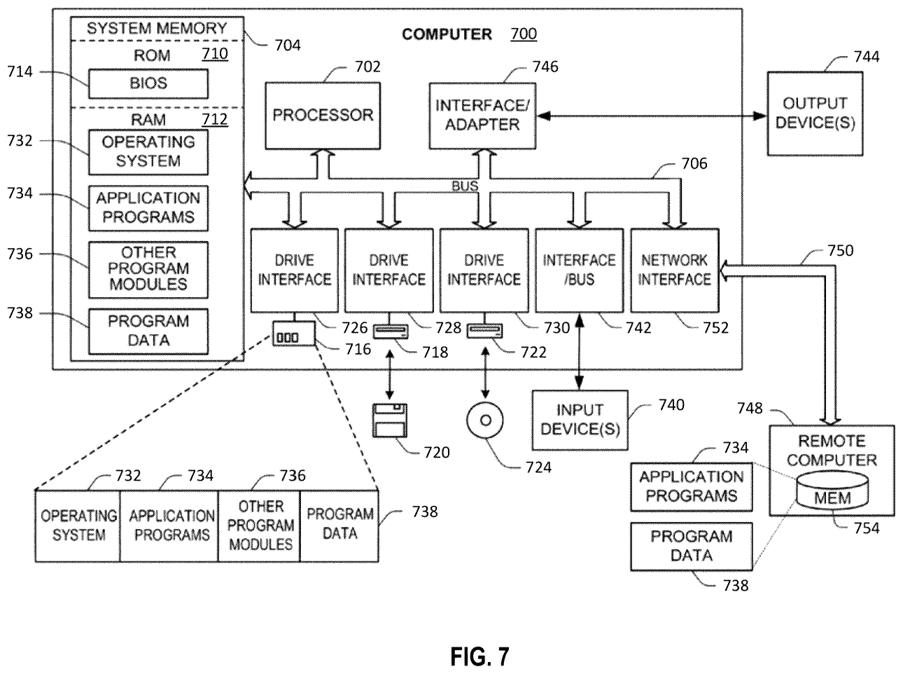

In view of the foregoing structural and functional description, those skilled in the art will appreciate that portions of the embodiments may be embodied as a method, data processing system, or computer program product. Accordingly, these portions of the present embodiments may take the form of an entirely hardware embodiment, an entirely software embodiment, or an embodiment combining software and hardware, such as shown and described with respect to the computer system of FIG. 7. Furthermore, portions of the embodiments may be a computer program product on a computer-readable storage medium having computer readable program code on the medium. Any non-transitory, tangible storage media possessing structure may be utilized including, but not limited to, static and dynamic storage devices, volatile and non-volatile memories, hard disks, optical storage devices, and magnetic storage devices, but excludes any medium that is not eligible for patent protection under 35 U.S.C. § 101 (such as a propagating electrical or electromagnetic signals per se). As an example and not by way of limitation, computer-readable storage media may include a semiconductor-based circuit or device or other IC (such, as for example, a field-programmable gate array (FPGA) or an ASIC), a hard disk, an HDD, a hybrid hard drive (HHD), an optical disc, an optical disc drive (ODD), a magneto-optical disc, a magneto-optical drive, a floppy disk, a floppy disk drive (FDD), magnetic tape, a holographic storage medium, a solid-state drive (SSD), a RAM-drive, a SECURE DIGITAL card, a SECURE DIGITAL drive, or another suitable computer-readable storage medium or a combination of two or more of these, where appropriate. A computer-readable non-transitory storage medium may be volatile, nonvolatile, or a combination of volatile and non-volatile, as appropriate.

Certain embodiments have also been described herein with reference to block illustrations of methods, systems, and computer program products. It will be understood that blocks and/or combinations of blocks in the illustrations, as well as methods or steps or acts or processes described herein, can be implemented by a computer program comprising a routine of set instructions stored in a machine-readable storage medium as described herein. These instructions may be provided to one or more processors of a general purpose computer, special purpose computer, or other programmable data processing apparatus (or a combination of devices and circuits) to produce a machine, such that the instructions of the machine, when executed by the processor, implement the functions specified in the block or blocks, or in the acts, steps, methods and processes described herein.

These processor-executable instructions may also be stored in computer-readable memory that can direct a computer or other programmable data processing apparatus to function in a particular manner, such that the instructions stored in the computer-readable memory result in an article of manufacture including instructions which implement the function specified. The computer program instructions may also be loaded onto a computer or other programmable data processing apparatus to cause a series of operational steps to be performed on the computer or other programmable apparatus to realize a computer implemented process such that the instructions which execute on the computer or other programmable apparatus provide steps for implementing the functions specified in flowchart blocks that may be described herein.

In this regard, FIG. 7 illustrates one example of a computer system 700 that can be employed to execute one or more embodiments of the present disclosure. Computer system 700 can be implemented on one or more general purpose networked computer systems, embedded computer systems, routers, switches, server devices, client devices, various intermediate devices/nodes or standalone computer systems. Additionally, computer system 700 can be implemented on various mobile clients such as, for example, a personal digital assistant (PDA), laptop computer, pager, and the like, provided it includes sufficient processing capabilities.

Computer system 700 includes processing unit 702, system memory 704, and system bus 706 that couples various system components, including the system memory 704, to processing unit 702. System memory 704 can include volatile (e.g. RAM, DRAM, SDRAM, Double Data Rate (DDR) RAM, etc.) and non-volatile (e.g. Flash, NAND, etc.) memory. Dual microprocessors and other multi-processor architectures also can be used as processing unit 702. System bus 706 may be any of several types of bus structure including a memory bus or memory controller, a peripheral bus, and a local bus using any of a variety of bus architectures. System memory 704 includes read only memory (ROM) 710 and random access memory (RAM) 712. A basic input/output system (BIOS) 714 can reside in ROM 710 containing the basic routines that help to transfer information among elements within computer system 700.

Computer system 700 can include a hard disk drive 716, magnetic disk drive 718, e.g., to read from or write to removable disk 720, and an optical disk drive 722, e.g., for reading CD-ROM disk 724 or to read from or write to other optical media. Hard disk drive 716, magnetic disk drive 718, and optical disk drive 722 are connected to system bus 706 by a hard disk drive interface 726, a magnetic disk drive interface 728, and an optical drive interface 730, respectively. The drives and associated computer-readable media provide nonvolatile storage of data, data structures, and computer-executable instructions for computer system 700. Although the description of computer-readable media above refers to a hard disk, a removable magnetic disk and a CD, other types of media that are readable by a computer, such as magnetic cassettes, flash memory cards, digital video disks and the like, in a variety of forms, may also be used in the operating environment; further, any such media may contain computer-executable instructions for implementing one or more parts of embodiments shown and described herein.

A number of program modules may be stored in drives and ROM 710, including operating system 732, one or more application programs 734, other program modules 736, and program data 738. In some examples, the application programs 734 can include control routines for the heaters 204, 402, and 412, the pumps 214 and 404, the high-speed camera 206 and any sub-programs thereof, and the program data 738 can include pressure and temperature readings, and images and videos collected via the high-speed camera 206. The application programs 734 and program data 738 can include functions and methods programmed to operate and maintain microfluidics experimental environments, and to capture images and videos of the experimental results, such as shown and described herein.

A user may enter commands and information into computer system 700 through one or more input devices 740, such as a pointing device (e.g., a mouse, touch screen), keyboard, microphone, joystick, game pad, scanner, and the like. For instance, the user can employ input device 740 to edit operational parameters of the heaters 204, 402, and 412, the pumps 214 and 404, the high-speed camera 206, as well as any other manual functions of the systems 200 and 400. These and other input devices 740 are often connected to processing unit 702 through a corresponding port interface 742 that is coupled to the system bus, but may be connected by other interfaces, such as a parallel port, serial port, or universal serial bus (USB). One or more output devices 744 (e.g., display, a monitor, printer, projector, or other type of displaying device) is also connected to system bus 706 via interface 746, such as a video adapter.

Computer system 700 may operate in a networked environment using logical connections to one or more remote computers, such as remote computer 748. Remote computer 748 may be a workstation, computer system, router, peer device, or other common network node, and typically includes many or all the elements described relative to computer system 700. The logical connections, schematically indicated at 750, can include a local area network (LAN) and/or a wide area network (WAN), or a combination of these, and can be in a cloud-type architecture, for example configured as private clouds, public clouds, hybrid clouds, and multi-clouds. When used in a LAN networking environment, computer system 700 can be connected to the local network through a network interface or adapter 752. When used in a WAN networking environment, computer system 700 can include a modem, or can be connected to a communications server on the LAN. The modem, which may be internal or external, can be connected to system bus 706 via an appropriate port interface. In a networked environment, application programs 734 or program data 738 depicted relative to computer system 700, or portions thereof, may be stored in a remote memory storage device 754.

Although this disclosure includes a detailed description on a computing platform and/or computer, implementation of the teachings recited herein are not limited to only such computing platforms. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models (e.g., software as a service (Saas, platform as a service (PaaS), and/or infrastructure as a service (IaaS)) and at least four deployment models (e.g., private cloud, community cloud, public cloud, and/or hybrid cloud). A cloud computing environment can be service oriented with a focus on statelessness, low coupling, modularity, and semantic interoperability.

Embodiments disclosed herein include:

A. A system for simulating flow within carbonate rock under reservoir conditions, the system comprising a substantially two-dimensional microfluidics device defining a flowpath therethrough, the substantially two-dimensional microfluidics device including a first wall comprising thin slices of carbonate rock, an opposing wall comprising transparent glass, a plurality of surrounding walls, and a throat defined within the flowpath to simulate flow through a porous structure, and a high-speed camera at or near the opposing wall and aimed at the first wall and the throat of the substantially two-dimensional microfluidics device, the high-speed camera operable to capture images and/or videos of fluid flow through the substantially two-dimensional microfluidics device.

B. A system for simulating flow out of carbonate rock under reservoir conditions, the system comprising a three-dimensional microfluidics device defining a flowpath therethrough, the three-dimensional microfluidics device including a plurality of walls including the flowpath, a packed bed of spheres within the plurality of walls and including a plurality of spheres of a carbonate rock composition, an interim fluid conduit in fluid communication with an outlet of the flowpath, and a secondary flow channel formed of transparent glass plates and in fluid communication with the interim fluid conduit to receive flow from the flowpath, and a high-speed camera at or near the secondary flow channel and aimed therethrough, the high-speed camera operable to capture images and/or videos of fluid flow out of the flowpath and packed bed and through the secondary flow channel.

C. A computer-implemented method for observing flow through a microfluidics device in reservoir conditions, the method comprising heating a flow environment within the microfluidics device to simulate a reservoir temperature within a flow environment of the microfluidics device, pumping a working fluid at a specified pressure to simulate a reservoir pressure within the flow environment, initiating, via a high-speed camera aimed at or near the flow environment, imaging of a flow through and/or out of the flow environment, and recording, via a computer-readable storage medium, images and/or videos obtained via the high-speed camera for analysis, wherein at least one wall of the microfluidics device includes a superhydrophobic coating applied thereon.

Each of embodiments A through C may have one or more of the following additional elements in any combination: Element 1: further comprising: a simulation chamber surrounding the substantially two-dimensional microfluidics device maintained at a specified pressure and a specified temperature. Element 2: further comprising: a heater mated to the simulation chamber and providing an influx of heat to the simulation chamber to maintain the substantially two-dimensional microfluidics device at the specified temperature. Element 3: further comprising: a pump in fluid communication with a fluid inlet of the substantially two-dimensional microfluidics device providing a working fluid at a specified pressure. Element 4: further comprising a source tank in fluid communication with the pump and containing a water-in-oil emulsion as the working fluid. Element 5: further comprising a particle emulsion tank in fluid communication with the source tank and containing one or more compositions of rock particles to be introduced to the working fluid. Element 6: further comprising a computing device including a processor and a computer-readable storage medium, wherein the computing device is operable to control the high-speed camera and operation of the substantially two-dimensional microfluidics device. Element 7: wherein at least one of the opposing wall and the plurality of surrounding walls includes a superhydrophobic coating applied thereto. Element 8: further comprising a light source installed at or near the secondary flow channel and providing a visible light source, an infrared light source, or a combination thereof to a working fluid in the secondary flow channel.

Element 9: wherein the packed bed of spheres is a matrix of sintered and/or compacted calcium-carbonate rock spheres. Element 10: further comprising: a fluid source in fluid communication with the three-dimensional microfluidics device and containing a working fluid therein; and a pump interposing the fluid source and the three-dimensional microfluidics device and maintaining a specified pressure within the three-dimensional microfluidics device.

Element 11: further comprising a particle emulsion tank in fluid communication with the fluid source and containing one or more compositions of rock particles to be introduced to the working fluid. Element 12: further comprising a source heater operably coupled to the fluid source and providing an influx of heat to the working fluid therein. Element 13: wherein at least one of the plurality of walls or the transparent glass plates includes a superhydrophobic coating applied thereto. Element 14: wherein the microfluidics device is a substantially two-dimensional microfluidics device including a first wall comprising thin slices of a carbonate rock.

Element 15: further comprising: exchanging the thin slices of carbonate rock of the first wall with a further set of thin slices of carbonate rock comprising a different carbonate rock. Element 16: wherein the microfluidics device is a three-dimensional microfluidics device including a matrix of packed rock spheres, a transparent secondary flow channel in fluid communication with the flow environment, and a light source at or near the transparent secondary flow channel. Element 17: further comprising switching the light source between visible light, infrared light, and/or a combination thereof while recording images and/or videos.

By way of non-limiting example, exemplary combinations applicable to A through C include: Element 1 with Element 2; Element 3 with Element 4; Element 4 with Element 5; Element 10 with Element 11; Element 10 with Element 12; Element 14 with Element 15; and Element 16 with Element 17.

The terminology used herein is for the purpose of describing particular embodiments only and is not intended to be limiting of the invention. As used herein, for example, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation used herein are merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third, etc.) is for distinction and not counting. For example, the use of “third” does not imply there must be a corresponding “first” or “second.” Also, if used herein, the terms “coupled” or “coupled to” or “connected” or “connected to” or “attached” or “attached to” may indicate establishing either a direct or indirect connection, and is not limited to either unless expressly referenced as such.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

The invention claimed is:

1. A system for simulating flow within carbonate rock under reservoir conditions, the system comprising:
a substantially two-dimensional microfluidics device defining a flowpath therethrough, the substantially two-dimensional microfluidics device including:
a first wall comprising thin slices of carbonate rock,
an opposing wall comprising transparent glass,
a plurality of surrounding walls, and
a throat defined within the flowpath to simulate flow through a porous structure; and
a high-speed camera at or near the opposing wall and aimed at the first wall and the throat of the substantially two-dimensional microfluidics device, the high-speed camera operable to capture images and/or videos of fluid flow through the substantially two-dimensional microfluidics device.

2. The system of claim 1, further comprising:
a simulation chamber surrounding the substantially two-dimensional microfluidics device maintained at a specified pressure and a specified temperature.

3. The system of claim 2, further comprising:
a heater mated to the simulation chamber and providing an influx of heat to the simulation chamber to maintain the substantially two-dimensional microfluidics device at the specified temperature.

4. The system of claim 1, further comprising:
a pump in fluid communication with a fluid inlet of the substantially two-dimensional microfluidics device providing a working fluid at a specified pressure.

5. The system of claim 4, further comprising a source tank in fluid communication with the pump and containing a water-in-oil emulsion as the working fluid.

6. The system of claim 5, further comprising a particle emulsion tank in fluid communication with the source tank and containing one or more compositions of rock particles to be introduced to the working fluid.

7. The system of claim 1, further comprising a computing device including a processor and a computer-readable storage medium, wherein the computing device is operable to control the high-speed camera and operation of the substantially two-dimensional microfluidics device.

8. The system of claim 1, wherein at least one of the opposing wall and the plurality of surrounding walls includes a superhydrophobic coating applied thereto.

9. A system for simulating flow out of carbonate rock under reservoir conditions, the system comprising:
a three-dimensional microfluidics device defining a flowpath therethrough, the three-dimensional microfluidics device including:
a plurality of walls including the flowpath,
a packed bed of spheres within the plurality of walls and including a plurality of spheres of a carbonate rock composition,
an interim fluid conduit in fluid communication with an outlet of the flowpath, and
a secondary flow channel formed of transparent glass plates and in fluid communication with the interim fluid conduit to receive flow from the flowpath; and
a high-speed camera at or near the secondary flow channel and aimed therethrough, the high-speed camera operable to capture images and/or videos of fluid flow out of the flowpath and packed bed and through the secondary flow channel.

10. The system of claim 9, further comprising a light source installed at or near the secondary flow channel and providing a visible light source, an infrared light source, or a combination thereof to a working fluid in the secondary flow channel.

11. The system of claim 9, wherein the packed bed of spheres is a matrix of sintered and/or compacted calcium-carbonate rock spheres.

12. The system of claim 9, further comprising:
a fluid source in fluid communication with the three-dimensional microfluidics device and containing a working fluid therein; and
a pump interposing the fluid source and the three-dimensional microfluidics device and maintaining a specified pressure within the three-dimensional microfluidics device.

13. The system of claim 12, further comprising a particle emulsion tank in fluid communication with the fluid source and containing one or more compositions of rock particles to be introduced to the working fluid.

14. The system of claim 12, further comprising a source heater operably coupled to the fluid source and providing an influx of heat to the working fluid therein.

15. The system of claim 9, wherein at least one of the plurality of walls or the transparent glass plates includes a superhydrophobic coating applied thereto.

16. A computer-implemented method for observing flow through a microfluidics device in reservoir conditions, the method comprising:
heating a flow environment within the microfluidics device to simulate a reservoir temperature within a flow environment of the microfluidics device;
pumping a working fluid at a specified pressure to simulate a reservoir pressure within the flow environment;
initiating, via a high-speed camera aimed at or near the flow environment, imaging of a flow through and/or out of the flow environment; and
recording, via a computer-readable storage medium, images and/or videos obtained via the high-speed camera for analysis,
wherein at least one wall of the microfluidics device includes a superhydrophobic coating applied thereon.

17. The computer-implemented method of claim 16, wherein the microfluidics device is a substantially two-dimensional microfluidics device including a first wall comprising thin slices of a carbonate rock.

18. The computer-implemented method of claim 17, further comprising:
exchanging the thin slices of carbonate rock of the first wall with a further set of thin slices of carbonate rock comprising a different carbonate rock.

19. The computer-implemented method of claim 16, wherein the microfluidics device is a three-dimensional microfluidics device including a matrix of packed rock spheres, a transparent secondary flow channel in fluid communication with the flow environment, and a light source at or near the transparent secondary flow channel.

20. The computer-implemented method of claim 19, further comprising switching the light source between visible light, infrared light, and/or a combination thereof while recording images and/or videos.
